(19) United States
(12) Patent Application Publication
(10) Pub. No.: US 2021/0156613 A1
(43) Pub. Date: May 27, 2021

(54) METHODS AND SYSTEMS FOR REMOVING NITROGEN FROM NATURAL GAS

(71) Applicant: Linde Engineering North America, Inc., Blue Bell, PA (US)

(72) Inventor: Kathryn Bigger, Mounds, OK (US)

(21) Appl. No.: 17/045,725

(22) PCT Filed: Apr. 8, 2019
(86) PCT No.: PCT/US2019/026300

§ 371 Date: Oct. 6, 2020

Related U.S. Application Data
(60) Provisional application No. 62/654,684, filed on Apr. 9, 2018.

(51) Int. Cl. F25J 3/02

(52) U.S. Cl. CPC F25J 3/0209; F25J 3/0257; F25J 3/0295; F25J 2200/04; F25J 2200/40; F25J 2290/40; F25J 2200/70; F25J 2205/04; F25J 2235/60; F25J 2240/12; F25J 2200/50

(57) ABSTRACT

Methods and systems for producing a product natural gas employing a natural gas liquids (NGL) recovery unit followed by removing nitrogen in a nitrogen rejection unit (NRU) operatively connected with the NGL recovery unit by a pressure management sub-system (PMSS). In one embodiment, the PMSS includes a first conduit fluidly connecting the top of a demethanizer column (or an existing conduit connected to the top of the demethanizer) to a separator, a second conduit fluidly connecting the separator to a pump, the pump connected to a distillation column in the NRU by a third conduit, in another embodiment, the PMSS includes a first conduit fluidly connecting an NGL expander to a separator, allowing natural gas vapors and nitrogen to be fed to the NRU column through a second conduit. Alternatively, the PMSS allows mixture from the NGL recovery unit expander to be fed directly via the first conduit to the NRU distillation column.

### CROSS-REFERENCE TO RELATED APPLICATIONS

This application is entitled to and claims the benefit of earlier filed provisional application Ser. No. 62/654,684, filed Apr. 9, 2018, under 35 U.S.C. § 119(e), which earlier filed provisional application is incorporated by reference herein in its entirety.

### BACKGROUND INFORMATION

### Technical Field

The present disclosure relates to methods and systems for managed pressure natural gas liquids (NGL) and nitrogen recovery in the hydrocarbon production field. In particular, the present disclosure relates to methods and systems featuring any one of a variety of NGL plants and any one of a variety of nitrogen rejection units operatively connected by a managed pressure sub-system so that the NGL plant and the nitrogen rejection plant may each be operated more efficiently, and/or with reduced capital expenditure.

### Background Art

Natural gas passing through transmission lines (conduits) frequently has an upper limit on the allowable nitrogen concentration therein, which typically ranges from a maximum of 2.0 to 4.0 mole percent nitrogen in the natural gas. Therefore, producers of natural gas containing higher concentrations of nitrogen must install facilities to reduce the nitrogen concentration in the natural gas to acceptable levels.

The removal of nitrogen from natural gas can be costly both with respect to CAPEX and OPEX (i.e., capital and operating expenditures). Some natural gas producers install a cryogenic nitrogen rejection unit, or cryogenic NRU, in series with other gas processing units such as a cryogenic NGL recovery unit.

More typically, the cryogenic NRU is installed sequentially with the cryogenic NGL recovery process and the natural gas feed is first cooled down from ambient temperature to cryogenic temperature to recover a natural gas liquid (C₂+) stream and a C₁ and lighter stream that is lean in C₂+ components. The C₁ and lighter stream that is lean in C₂+ components is warmed back up to ambient temperature, compressed, and then fed to the NRU where it is cooled down from ambient temperature to cryogenic temperature to remove (or reject) the nitrogen and form a nitrogen-lean C and lighter stream that is lean in C₂+ components. This is followed by warming up the nitrogen-lean C and lighter stream that is lean in C₂+ components back up to ambient temperature. In some cases, the order of the unit operations is reversed such that the nitrogen rejection step is first and the step of forming a natural gas liquid (C₂+) stream and a C₁ and lighter stream that is lean in C₂+ components is performed second.

It is advantageous to integrate the nitrogen rejection process into the cryogenic NGL recovery process such that cooling down the natural gas feed to cryogenic temperature, and then warming up the natural gas product to ambient temperature is only done once rather than twice as when the units are installed in series. This has the potential to save OPEX in the form of reduced energy consumption for refrigeration by only needing to cool down the natural gas one time and reduced CAPEX by reducing the size of the refrigeration compression equipment that must be installed.

One of the more common methods for cryogenic NGL recovery is to use the Gas Subcooled Process (GSP) or variants thereof, of which the main equipment includes a refrigeration system which is typically propane-based, a turboexpander, a “subcooler” heat exchanger, and a demethanizer column. If only propane and heavier (C₃+) components are being recovered in the NGL recovery unit, the “demethanizer” column is a “deethanizer” column. The demethanizer, as described herein could also describe a deethanizer if the NGL Recovery unit is operating in ethane rejection mode. The term “C₂+ components” or “C₂+ hydrocarbons” as described herein could also describe “C₃+ components” or “C₃+ hydrocarbons”, particularly if the NGL recovery unit column is operating as a deethanizer.

If an NRU is to be integrated into a GSP NGL recovery process or other such cryogenic process that includes a demethanizer, it is advantageous to send cold demethanizer overhead directly to the NRU, before the demethanizer overhead (predominantly C₁ natural gas product) is warmed back up to ambient temperature. After the nitrogen is removed from the natural gas to acceptable levels, the natural gas product can then be warmed up to ambient temperature and compressed to pipeline transmission pressure. In this manner, the natural gas liquids (C₂+) and nitrogen are removed from the natural gas to form a predominantly C₁ natural gas product in one cycle of cooling down the feed natural gas and reheating the predominantly C₁ natural gas product.

One of the challenges in such a configuration for an integrated GSP and NRU plant is that normally it is desirable to operate the demethanizer at a pressure lower than the distillation column within the NRU. In the apparent configuration, in order to integrate the GSP and NRU, the demethanizer column must be operated at a pressure higher than desired for the GSP unit, such that the demethanizer overhead stream feeding the NRU is at sufficient pressure to feed the NRU column.

The methods and systems of the present disclosure address this issue allowing the demethanizer to be operated at more optimum pressure, which is usually lower than, but could also be equal to, or just above the operating pressure of the NRU column.

The methods and systems of the present disclosure therefore operatively connect the natural gas liquids recovery and nitrogen rejection units while allowing the demethanizer column and the NRU column to each operate at its' optimum pressure, providing cost savings in both reduced capital expenditures and reduced operating expenditures when compared to sequential natural gas recovery and NRU units.

Various efforts in this area may be exemplified by U.S. Pat. Nos. 9,487,458; 9,726,426; and 9,816,752. However, none of these documents mention a pressure management sub-system operatively connecting an NGL recovery unit and an NRU, as taught by the present disclosure.

As may be seen, current practice may not be adequate for all circumstances, and may result in higher demethanizer pressures, which result in higher power requirements, and/or lower natural gas recovery. There remains a need for more robust managed pressure methods and systems. The methods and systems of the present disclosure are directed to these needs.

### SUMMARY

In accordance with the present disclosure, methods and systems are described which reduce or overcome many of the faults of previously known methods and systems. The methods and systems of the present disclosure allow the nitrogen rejection unit to be integrated, i.e., operatively connected, into the natural gas liquids recovery unit with little or no negative impact on the operation of the natural gas liquids recovery unit. The methods and systems of the present disclosure result in reduced refrigeration horsepower in the nitrogen rejection unit versus the sequential natural gas liquids recovery and nitrogen rejection unit processing, resulting in lower capital expenditures and operating expenditures versus sequential natural gas liquids recovery and nitrogen rejection units.

A first aspect of the disclosure are methods, one method embodiment comprising (or consisting essentially of, or consisting of):

(a) routing one or more raw natural gas streams to a natural gas processing plant, the natural gas processing plant comprising an NGL recovery unit including a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3-4 mole percent;

(b) removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form the reject nitrogen stream and the product natural gas;

(c) operating the demethanizer column at a pressure lower than, equal to, or just above the NRU distillation column by managing a pressure relationship between the demethanizer column and the NRU distillation column using a pressure management sub-system (PMSS) comprising a separator, a pump and an expansion valve, comprising:
(i) routing at least a portion of demethanizer column overhead to one or more heat exchangers within the NRU to partially or wholly condense the demethanizer overhead feeding the heat exchanger or exchangers;
(ii) routing the partially or wholly condensed demethanizer overhead to a separator to separate any remaining vapor from the liquid;
(iii) pumping the liquid stream from the separator into a lower section of the NRU distillation column using the pump;
(iv) combining any uncondensed demethanizer overhead vapor from the separator with the NRU distillation column bottoms stream, after the NRU distillation column bottoms has been reduced in pressure via an expansion valve, whereby the combined stream can now be called the natural gas product;
(v) routing the natural gas product to one or more heat exchangers within the NRU to reheat the stream to a temperature that is similar to the demethanizer overhead temperature; and
(vi) routing the natural gas product leaving the NRU to one or more heat exchangers within the NGL recovery unit to heat the natural gas product to ambient temperature.

In certain embodiments the raw natural gas stream may be routed to the NGL recovery unit prior to the NRU, while in certain other embodiments the raw natural gas stream may be routed to the NRU prior to the NGL recovery unit. In certain embodiments the NGL recovery unit may comprise a gas-subcooled process (GSP), wherein the raw natural gas is routed through one or more heat exchangers to produce one or more subcooled raw natural gas feed streams to the demethanizer column. In certain embodiments the PMSS may comprise one or more redundant components, for example, two or more pumps arranged in parallel flow relationship, or two or more separators arranged in parallel flow relationship. In certain embodiments, components of the PMSS may be arranged in series flow relationship, for example, two or more separators arranged in series, where liquid separated from upstream separators is caused to flow into a downstream separator. Embodiments with mixed parallel and series flow are also contemplated, for example, an arrangement of four separators where first and second separators are arranged in parallel with each other, third and fourth separators are arranged in parallel with each other, and where the first is in series with the third, and the second is in series with the fourth. In certain embodiments, cooling and condensing of the demethanizer overhead in the NRU and reheating of the cold natural gas product from the NRU can take place in one heat exchanger, whereas in other embodiments one or both of these heat exchanges can take place in two or more heat exchangers. Embodiments are also contemplated where the demethanizer overhead and/or the cold natural gas product can also exchange heat with other streams in the NRU, and such heat exchange can occur in one or more heat exchangers within the NRU.

A second aspect of the disclosure are systems, one system embodiment comprising (or consisting essentially of, or consisting of):

(a) an NGL recovery unit including a demethanizer column;

(b) an NRU including a distillation column; and

(c) a pressure management sub-system (PMSS) operatively and fluidly connected to the NGL recovery unit and the NRU, the PMSS comprising at least first through seventh conduits (inclusive), a separator, a pump, and an expansion valve, and further comprising:
(i) the first conduit configured to route at least a portion of demethanizer column overhead to one or more heat exchangers within the NRU and then to the separator;
(ii) the second conduit to route a liquid stream from the separator to a pump;
(iii) the third conduit to route liquid from the separator into a lower section of the NRU distillation column using the pump;
(iv) the fourth conduit configured to route at least a portion of reduced nitrogen NRU distillation column bottoms to the expansion valve and the fifth conduit to route this portion of the NRU distillation column bottoms to the heat exchanger or exchangers;
(v) the sixth conduit to blend any vapor leaving the separator with the NRU distillation column bottoms downstream of the expansion valve and upstream of the heat exchanger or exchangers to form the natural gas product; and
(vi) the seventh conduit to route the natural gas product to the NGL recovery unit heat exchanger network.

The term “NGL recovery unit” is to be interpreted to include, but is not limited to, gas subcooled processes (GSP) and non-gas subcooled processes, and “NGL recovery unit” can also refer to other processes, such as but not limited to, the Recycle Split Vapor (RSV) process and the CryoPlusm process. As used herein, “natural gas product” means a composition consisting essentially of methane and having from about 2 to about 4 mole percent nitrogen therein, that is substantially devoid of C₂+ hydrocarbon components, substantially devoid of water (H₂O) and may be substantially devoid of CO₂. As used herein “nitrogen rejection unit” and NRU mean a unit employing cryogenic separation techniques, unless otherwise specified to include other separation techniques, such as membrane separation and adsorption media separation. As used herein “pressure management sub-system” or PMSS means a component or combination of components (as detailed herein) operatively and fluidly connecting one or more NGL recovery units to one or more NRUs, and functioning to raise pressure of a distillation column in an NRU to be above, equal, or just below the pressure of a demethanizer column in an NGL recovery unit, or reduce a pressure of a demethanizer column in an NGL recovery unit to be below, equal, or just above a pressure of a distillation column in an NRU. As used herein a “receiver” is a pipeline, storage tank, underground storage cavern, tank truck, or any combination thereof.

In certain embodiments a logic device may be provided to control the pressure management sub-system, and the logic device may be configured to be operated and/or viewed from a Human/Machine Interface (HMI) wired or wirelessly connected to the logic device. Certain embodiments may include one or more audio and/or visual warning devices configured to receive communications from the logic device upon the occurrence of a pressure rise (or fall) in a sensed pressure above (or below) a set point pressure, or a change in concentration of one or more sensed concentrations or temperatures, or both, above one or more set points. The occurrence of a change in other measured parameters outside the intended ranges may also be alarmed in certain embodiments. Other measured parameters may include, but are not limited to, liquid flow rate, vapor flow rate, multiphase fluid flow rate, gas flow rate, and density of any of these.

Certain method and system embodiments of this disclosure may comprise starting up or shutting down one, more than one, or all operational equipment of a NGL recovery unit, a PMSS, and/or an NRU using one or more logic devices and the pressure management sub-system (for example as dictated by a client, law, or regulation), and in the case of shutting down, upon the occurrence of an adverse event. As used herein, the term “operational equipment” includes, but is not limited to, compressors, expanders, heat exchangers, separators, conduits, pumps, valves, and columns. “Adverse event” may include, but is not limited to, the presence of explosive vapors, H₂S, and/or pressure inside one or more operational equipment components considered unsafe, and which the pressure management sub-system is designed to shutoff above a maximum set point pressure (which may be independently set for each operational unit or conduit). In certain embodiments this may correspond with the detection of pressure by the pressure management sub-system above a maximum set point pressure. “Non-adverse event” time periods are interchangeable with “safe operating conditions” and “safe working conditions.”

Certain method and system embodiments of this disclosure may operate in modes selected from the group consisting of automatic continuous mode, automatic periodic mode, and manual mode. In certain embodiments the one or more operational equipment may include prime movers selected from the group consisting of pneumatic, electric, fuel, hydraulic, and combinations thereof.

In certain embodiments, pressure (P) and/or temperature (T) may be sensed inside the demethanizer column, the NRU distillation column, separators, expander exits, expansion valve inlet and outlets, and the like. Different pressure management sub-systems within a set of pressure management sub-systems may have different sensor strategies, for example, a mass flow sensor for one pressure management sub-system sensing mass flow inside the pressure management sub-system, another sensing mass flow inside a second pressure management sub-system. All combinations of sensing T, P, and/or mass flow inside and/or outside one or more pressure management sub-systems are disclosed herein and considered within the present disclosure.

Pressure management sub-systems may include pressure management components and associated components, for example, but not limited to pressure control devices (backpressure valves), pressure relief devices (valves or explosion discs), expansion valves, pipes, conduits, vessels, towers, tanks, mass flow meters, temperature and pressure indicators, heat exchangers, pumps, compressors, and expanders. With respect to “pressure management”, when referring to a PMSS, the managed pressure may, in some embodiments, be from about 100 psia (690 kPa) or less to about 1,200 psia (8,275 kPa) or greater; alternatively greater than about 200 psia (1,380 kPa); alternatively greater than about 300 psia (2,070 kPa); alternatively greater than about 400 psia (2,760 kPa), or greater than about 500 psia (3,450 kPa). For example, managed pressures may range from about 200 to about 800 psia (about 1,380 to about 5,520 kPa); or from about 250 to about 750 psia (about 1,725 to about 5,175 kPa); or from about 300 to about 700 psia (about 2,070 to about 4,830 kPa); or from about 250 to about 500 psia (about 1,725 to about 3,450 kPa); or from about 200 to about 450 psia (about 1,380 to about 3,105 kPa); or from about 300 to about 600 psia (about 2,070 to about 4,140 kPa); or from about 400 to about 600 psia (about 2,760 to about 4,140 kPa); or from about 300 to about 500 psia (about 2,070 to about 3,450 kPa); or from about 400 to about 800 psia (about 2,760 to about 5,520 kPa); or from about 500 to about 700 psia (about 3,450 to about 4,830 kPa). All ranges and sub-ranges (including endpoints) between about 100 psia (about 690 kPa) and about 1,200 psia (about 8,275 kPa) are considered explicitly disclosed herein. As used herein with respect to pressure, “about” means +/−10 psia (+/−69 kPa) for pressure point values equal to or below 300 psia (2,070 kPa), and +/−50 psia (+/−345 KPa) above 300 psia (2,070 KPa).

These and other features of the methods and systems of the present disclosure will become more apparent upon review of the brief description of the drawings, the detailed description, and the claims that follow. Methods of making natural gas products using one of the systems of the present disclosure are considered within the present disclosure. It should be understood that wherever the term “comprising” is used herein, other embodiments where the term “comprising” is substituted with “consisting essentially of” are explicitly disclosed herein, and vice versa. It should be further understood that wherever the term “comprising” is used herein, other embodiments where the term “comprising” is substituted with “consisting of” are explicitly disclosed herein, and vice versa. Moreover, the use of negative limitations is specifically contemplated; for example, certain sensors may trigger audible alarms but not visual alarms, and vice versa. In certain embodiments the refrigerant may not include more than a trace of CO₂. As another example, a pressure management sub-system may be devoid of a pump, or may be devoid of a separator.

### BRIEF DESCRIPTION OF THE DRAWINGS

The manner in which the objectives of this disclosure and other desirable characteristics can be obtained is explained in the following description and attached drawings in which:

FIGS. 1A and 1B are high-level schematic block diagram representations of methods and systems in accordance with the present disclosure;

FIGS. 2, 3, and 4 are schematic process flow diagrams of three embodiments of methods and systems in accordance with the present disclosure;

FIGS. 5 and 6 are highly schematic views of two other method and system embodiments in accordance with the present disclosure; and

FIGS. 7A and 7B; 8A and 8B; and 9A and 9B are schematic logic diagrams of three method embodiments in accordance with the present disclosure.

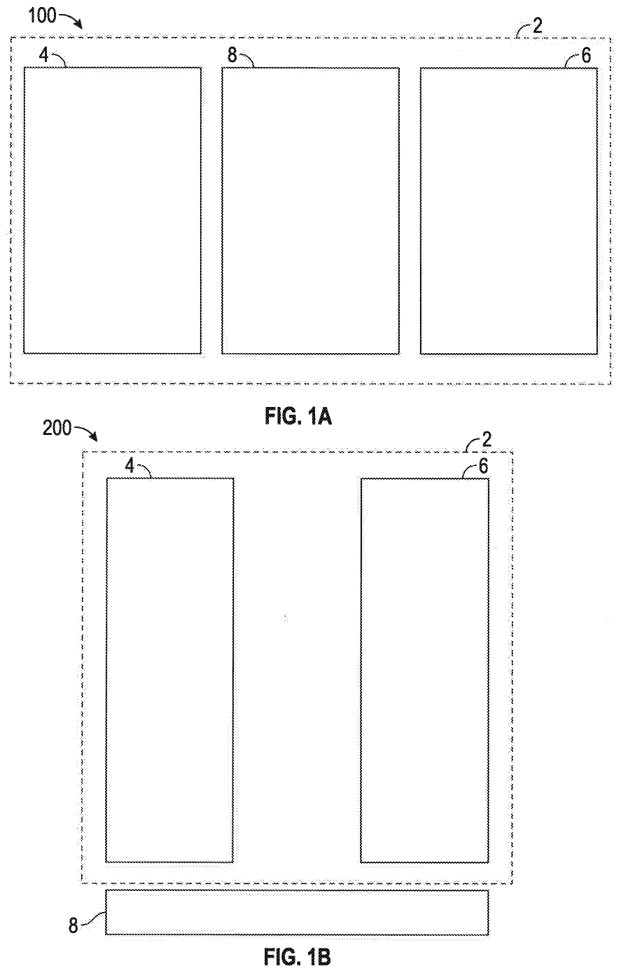

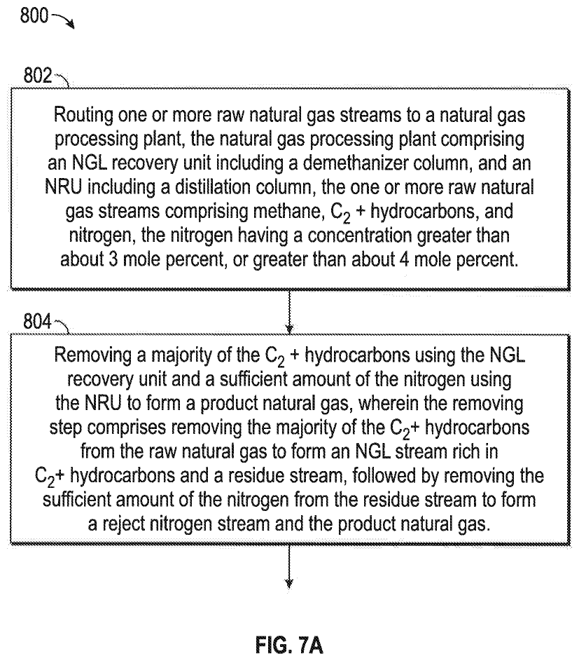

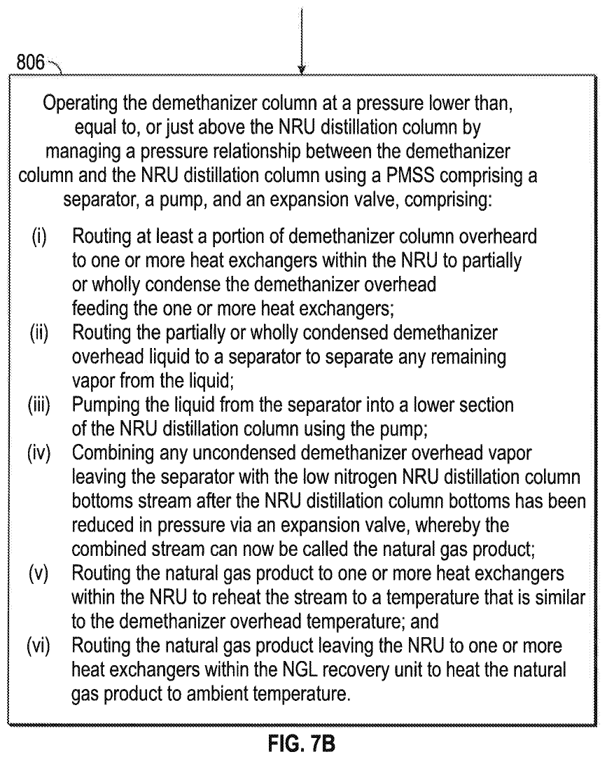

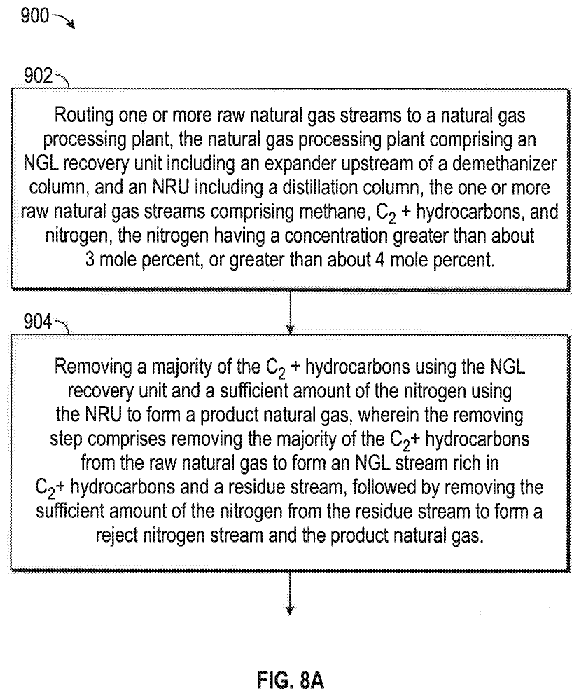

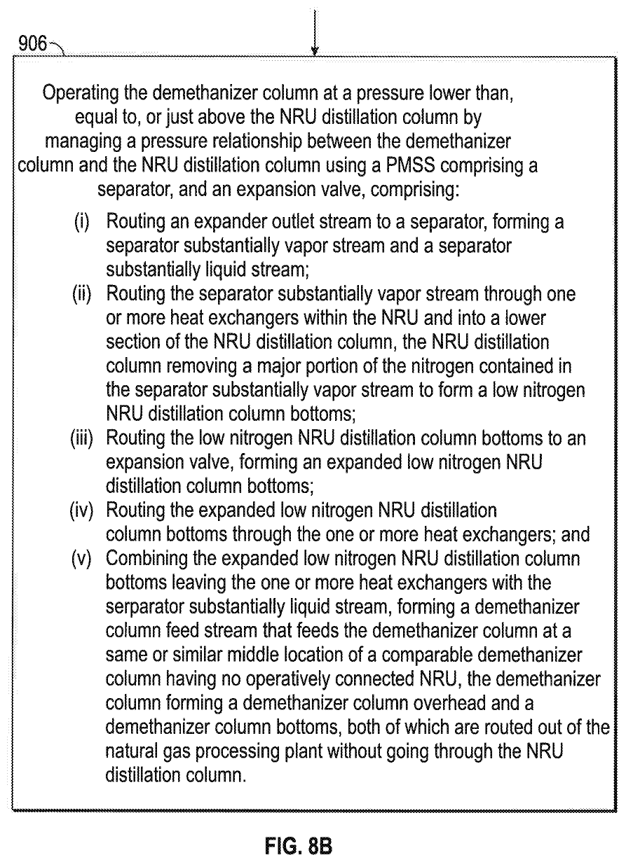

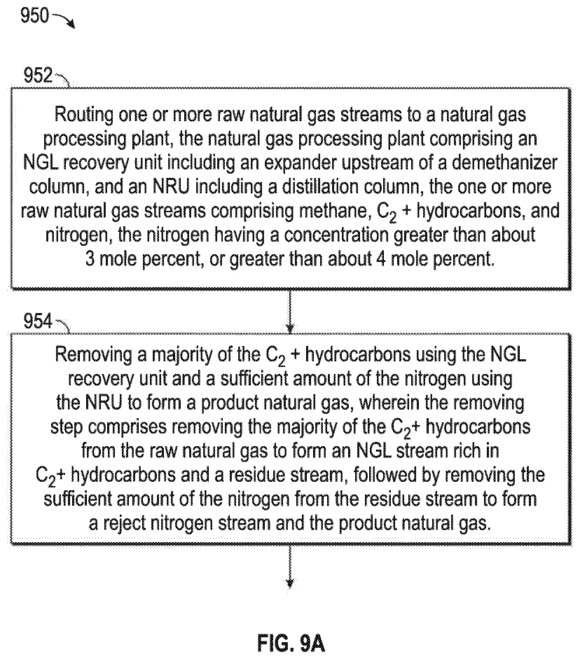

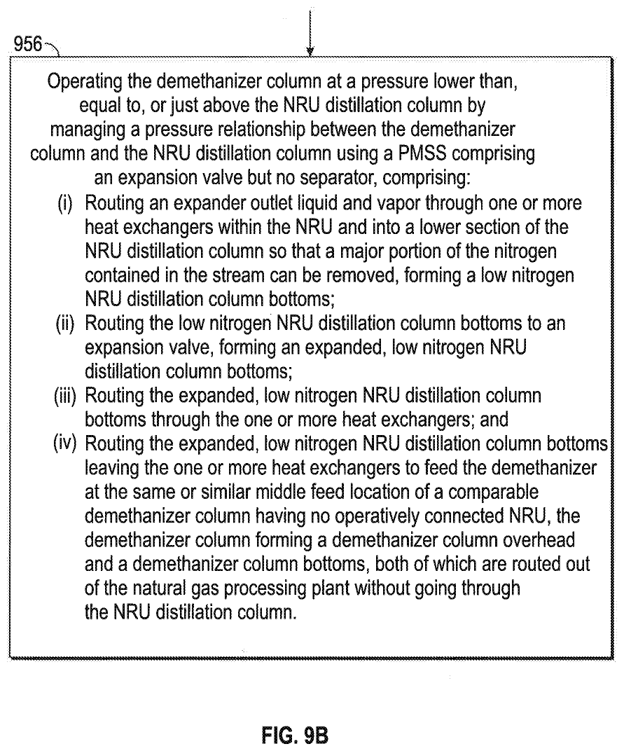

Itis to be noted, however, that the appended drawings of FIGS. 1A, 1B, and 2-6 are not to scale, and illustrate only typical system and method embodiments of this disclosure. Furthermore, FIGS. 7A and 7B; 8A and 8B; and 9A and 9B illustrate only three of many possible methods in accordance with this disclosure. Therefore, the drawing figures are not to be considered limiting in scope, for the disclosure may admit to other equally effective embodiments. Identical reference numerals are used throughout the several views for like or similar elements.

### DETAILED DESCRIPTION

In the following description, numerous details are set forth to provide an understanding of the disclosed apparatus, combinations, and processes. However, it will be understood by those skilled in the art that the apparatus, systems, and processes disclosed herein may be practiced without these details and that numerous variations or modifications from the described embodiments may be possible. All technical articles, U.S. published and non-published patent applications, standards, U.S. patents, U.S. statutes and regulations referenced herein are hereby explicitly incorporated herein by reference, irrespective of the page, paragraph, or section in which they are referenced. Where a range of values describes a parameter, all sub-ranges, point values and endpoints within that range or defining a range are explicitly disclosed herein. All percentages herein are by weight unless otherwise noted.

As mentioned herein, one of the challenges in operating NGL recovery units and NRU units that are operably connected is that normally it is desirable to operate the demethanizer column of the NGL recovery unit at a pressure lower or perhaps equal to or just slightly above the distillation column within the NRU. In configurations considered outside of those presently disclosed, in order to operatively connect the GSP and NRU, the demethanizer column must be operated at a pressure higher than desired for the GSP unit, such that the demethanizer column overhead stream feeding the NRU is at sufficient pressure to feed the NRU distillation column after passing through intervening equipment such as heat exchangers and control valves. The methods and systems of the present disclosure address this issue by allowing the demethanizer column to be operated at more optimum pressure, which is usually lower than operating pressure of the NRU distillation column. Methods and systems of the present disclosure therefore operatively connect the natural gas liquids recovery and nitrogen rejection units while allowing the demethanizer column and the NRU distillation column to each operate at its' optimum pressure, providing cost savings in both reduced capital expense and reduced operating expense when compared to sequential natural gas recovery and NRU units.

Methods and systems of the present disclosure enable virtually any NGL recovery process (whether using GSP or not) and NRU combination to potentially more efficiently produce natural gas product, with potentially minimal to no modifications to the NGL recovery unit or the NRU. In certain embodiments, all that may be required to form one embodiment of a PMSS are additional conduits and valves, and perhaps an expansion (Joule-Thompson, or “JT”) valve.

As described in more detail herein with reference to the various drawing figures, methods and systems of the present disclosure may be comprised of two main process units or “plants” operatively connected by a pressure management sub-system (PMSS). The first process unit or plant is termed a natural gas liquids (NGL) recovery unit, which functions to remove C₂+ (or C₃+) and heavier components from raw natural gas, producing a C₂+ (or C₃+) and heavier predominantly liquid composition, and a predominantly gas composition comprised predominantly of Ct and lighter components, including nitrogen, argon, helium, and the like. The NGL recovery unit need not be a GSP; any NGL recovery process may be used with any embodiment of the present disclosure described herein, as long as the method or process includes a demethanizer or deethanizer operating (or in the case of systems, configured to operate) at temperatures below ambient; ambient temperature may range from about 32° F. to about 100° F. (0° C. to about 38° C.), or from about 50° F. to about 77° F. (10° C. to about 25° C.). The second process unit or plant is termed a nitrogen rejection unit (NRU), which functions to remove nitrogen, argon, helium, and the like from the predominantly gas composition comprised predominantly of Ct and lighter components, including nitrogen, argon, helium, and the like, using a distillation column, producing a distillation column overhead composition comprised predominantly of nitrogen, argon, helium, and the like, and a bottoms composition comprised predominantly of methane, plus minor amounts of C₂+ heavier components and minor amounts of nitrogen, argon, helium, and the like. As a convenience, for the remainder of this document, the overhead composition comprised predominantly of nitrogen, argon, helium, and the like will simply be referred to as the “nitrogen composition”, “nitrogen stream”, or “nitrogen reject stream”, while the bottoms composition comprised predominantly of methane, plus minor amounts of C₂+ heavier components and minor amounts of nitrogen, argon, helium, and the like will simply be referred to as the “methane product”, or the “product natural gas”, or the “methane product gas.” In other words, the gas that will have a nitrogen concentration suitable for “natural gas transmission lines.” Moreover, if only propane and heavier are being recovered in the NGL recovery unit, the “demethanizer column” herein may be deemed a deethanizer column; therefore, in all instances herein where the terms “demethanizer” and “demethanizer column” are used, this includes the terms “deethanizer” and “deethanizer column.” The location of the NGL recovery unit relative to the NRU is determined based on the desired application for the system. Factors such as process control, terrain, availability and size of line pipe or other conduits, availability and size of separators, pumps, and expansion valves, and desired pressures and pressure control mode, among others, can impact the placement of the NGL recovery unit and the NRU, and the configuration of the PMSS.

In certain methods and systems of this disclosure, the nitrogen molar concentration of the demethanizer overhead may be below about 20 mole percent, or below about 15 mole percent, or below about 10 mole percent, or below about 9 mole percent, or below about 8 mole percent, or below about 7 mole percent, or below about 6 mole percent, or below about 5 mole percent, or below about 4 mole percent, and the nitrogen molar concentration of the product natural gas may range from about 2 to about 4 mole percent, or from about 2.0 to about 4.0 mole percent. For example, studies have shown that the residue gas (demethanizer overhead) contained 6.2 mole percent nitrogen and the natural gas residue downstream of the integrated natural gas liquids recovery and nitrogen rejection unit contained 3.0 mole percent nitrogen.

The pressure of the available raw natural gas and the specific configuration of the PMSS largely define the type of managed pressure operation capabilities of each method and system embodiment. Redundancy of components in the PMSS may allow for extended service periods and mitigates risk of downtime due to component failure. An example would be a pressure control device plugging with frozen material, or a pump failure, or a separator taken out of service for inspection. In this case, isolating the failed or to be inspected component and enabling another one allows for continued operations, and enables evaluation and/or modification of the operational parameters to minimize the risk of failure of the new or parallel components in use.

The methods and systems of this present disclosure may be used for new greenfield applications, where one or both of the NGL recovery unit and the NRU are custom designed together to be operatively and fluidly connected during operation. It is also contemplated to design the NGL recovery unit and NRU to be able to operate in dual modes, where in the first mode the NGL recovery unit is integrated with the NRU, and the second mode where one or both of the units may operate independently from each other, in other words, where either one or both of the NGL recovery unit and the NRU may operate without requiring the other unit to be in operation.

Advantageously, most of the components of methods and systems of the present disclosure may alternatively be sourced from existing pieces of equipment used in the oil and gas industry. Some of the components of the systems of the present disclosure may be based on existing equipment, some of which may require modification to reconfigure the equipment for integrated operation between the NGL recovery unit and the NRU. The installation of methods and systems of the present disclosure on the NGL recovery unit and/or the NRU are expected to require minimal interfacing. It may be possible to design a retrofitted system that requires no modifications to the demethanizer or NRU distillation column, although the designer may consider modest changes, for example, substituting packing, grids, or other new internals for existing internals. New equipment to complete the integration of the NGL recovery unit and NRU may include components of the PMSS, new components in the NGL recovery unit, new components in the NRU, and/or a completely new NGL recovery unit or NRU.

Methods and systems of the present disclosure may be operated using hydraulic, electric, geothermal, pneumatic, or combustion power, or combination of one or more of these. One possible configuration may employ electric power to operate a motor for a pump of the PMSS (which motor may be variable speed or non-variable speed) and combustion power to operate the NGL recovery unit compressor(s) and NRU compressor(s). In certain embodiments, expanders and compressors may share a shaft. Power supplies may have redundant and/or back up power supply. In certain embodiments, electric power may require installation of an additional battery unit, possibly including solar panels for backup power. In certain embodiments, a plant may have one or more hydrocarbon-powered electric generators, and these units may provide electric power, and backup power may be provided by an uninterruptible power supply (UPS) battery system.

Certain embodiments may include 1) low power electric connections for data transmission for sensors (e.g., pressure, temperature, mass flow indicators, among others); and 2) electric cable to provide power for operating valves and components of the PMSS NGL recovery unit, and NRU. With respect to data connection/integration, in certain embodiments control signals for the components of the systems of the present disclosure, as well as parameters measured or captured by the system's sensors (e.g., pressures, temperatures, fluid flow rates and density, and the like) may be transmitted to and from an operator room or control room from and to the PMSS, the NGL recovery unit, and the NRU.

Referring now to the drawing figures, FIGS. 1A and 1B are schematic, highly simplified views of two system embodiments 100 and 200 in accordance with the present disclosure. Embodiments 100 and 200 each include a natural gas plant 2, denoted by the dashed line 2, an NGL recovery unit 4, an NRU 6, and a PMSS 8, however, embodiment 100 emphasizes that PMSS 8 may be located in and be considered a part of natural gas plant 2, for example when a natural gas plant is newly constructed, or when modifying a natural gas plant that had only the NGL recovery unit 4 and an NRU 6, whereas embodiment 200 emphasizes that PMSS 8 may be located outside of natural gas plant 2, for example when PMSS 8 is truck-mounted or ship-mounted, or when it simply makes sense to have the PMSS outside of the natural gas plant.

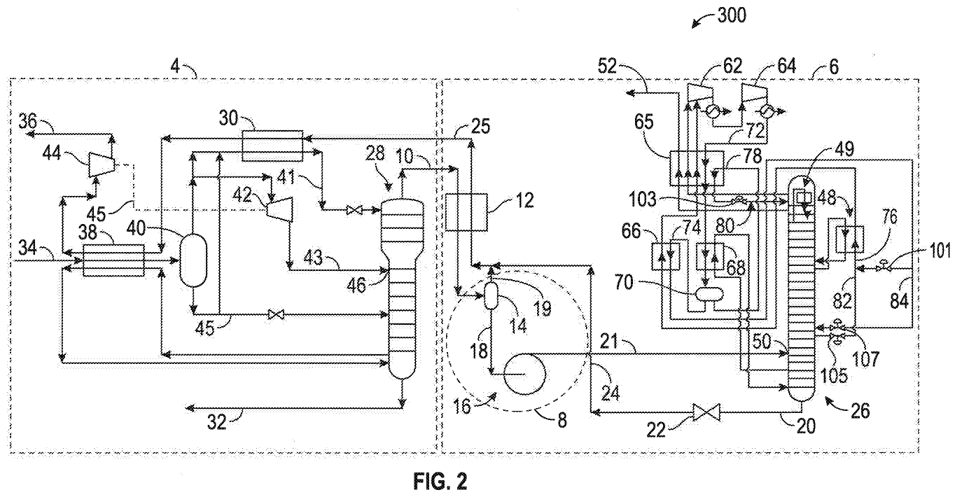

FIGS. 2, 3, and 4 are schematic process flow diagrams of three method and system embodiments 300, 400, and 500, respectively. In each of embodiments 300, 400, and 500, NGL recovery unit 4 includes a demethanizer column 28 producing a NGL stream that exits the plant through a demethanizer bottoms conduit 32; a raw natural gas feed conduit 34 that is cooled by heat exchange with various streams in NGL recovery unit primary heat exchanger 38 and then is routed to NGL recovery unit main separator 40. An NGL recovery unit expander 42 receives a portion of the overhead vapor stream from NGL recovery unit separator 40, and produces an expanded, cooled raw natural gas stream that is routed to demethanizer column 28 through a feed conduit 43 at a middle location 46 in demethanizer column 28 in embodiment 300 only. In embodiments 400 and 500, conduit 43 directs the raw natural gas to NRU 6 as is discussed herein. The remaining portion of the vapor overhead stream from separator 40 is routed to the heat exchanger known as the subcooler 30. Subcooler 30 is not required in all embodiments, however. Each of embodiments 300, 400, and 500 further includes an NGL recovery unit product booster compressor 44, which may or may not be mechanically connected to NGL recovery unit expander 42 via a common shaft 45. A natural gas product conduit 36 is further provided to route the stream to the downstream equipment (not shown), typically equipment to compress up to gas transmission line pressure. In other embodiments, the booster compressor 44 may instead be located upstream of heat exchanger 38, in what is known as the “pre-boost” configuration; in this case, conduit 36 routes the natural gas product from heat exchanger 38 directly to downstream operations without a booster compression step. Conduits 41 and 45 route the remaining raw natural gas streams to feed various locations in the demethanizer column 28.

The NGL recovery unit heat exchangers 38 and 30 in embodiments 300, 400, and 500 as illustrated schematically in FIGS. 2, 3, and 4, respectively, represent a typical configuration of the heat exchanger network within an NGL recovery unit. However, those of ordinary skill in this art will readily understand that other heat exchanger network configurations may be used in the NGL recovery unit, including but not limited to, combining heat exchangers 30 and 38 into one unit, or splitting heat exchangers 30 and 38 into three or more separate units. Additional streams and/or heat exchangers not illustrated in embodiments 300, 400, and 500 may also be present in the heat exchanger network, when advanced heat integration and/or external refrigeration is employed in the NGL recovery unit.

In each of embodiments 300, 400, and 500, NRU 6 includes an NRU distillation column 26; an NRU first heat exchanger 12; an NRU feed conduit 21 feeding to a near bottom location 50 of NRU distillation column 26; an NRU distillation column bottoms conduit 20 routing NRU distillation column bottoms to an expansion valve 22; an expanded NRU bottoms conduit 24; a nitrogen product conduit 52; refrigeration compressors 62, 64 and associated intercooler and aftercooler; an NRU second heat exchanger 65; an NRU third heat exchanger 66; an NRU column reboiler 68; a refrigerant vessel 70; and a side condenser 48 in addition to an overhead condenser 49, however, side condenser 48 is not necessary in all embodiments. Side condenser 48 may be advantageous in that it may reduce load on refrigeration compressor(s) 62, 64.

Each embodiment 300, 400, and 500 illustrated schematically in FIGS. 2, 3, and 4, respectively, includes an NRU refrigeration loop that may comprise the conduits described in Table 1.

TABLE 1
Conduits in NRU Refrigeration Loop

| Conduit | Description | Stream Type |
|---|---|---|
| 72 | HP Refrigerant to heat exchangers 65 and 68 | Hot Stream |
| 74 | HP Refrigerant to heat exchanger 66 | Hot Stream |
| 76 | MP Refrigerant to heat exchangers 48, 66, and 65 | Cold Stream |
| 78 | HP Refrigerant to heat exchanger 65 | Hot Stream |
| 80 | LP Refrigerant to heat exchangers 49 and 65 | Cold Stream |
| 82 | Refrigerant Makeup via valve 105 | Intermittent |
| 84 | Refrigerant Blowdown via valve 107 | Intermittent |

The refrigeration loop illustrated schematically in FIGS. 2, 3, and 4 and the conduits described in the table above comprise an example of a typical refrigeration loop for an NRU. Other refrigeration loop configurations are possible within the NRU, depending upon the specific design parameters. In certain embodiments, the refrigerant may primarily comprise methane but may also comprise the following components in small (from about 0.1 to about 1 mole percent) to trace amounts (about 0.1 mole percent or less, but more than 0 mole percent): nitrogen, ethane, propane, butanes, and minute quantities of CO₂. In conduit 72, ambient temperature high pressure (HP) refrigerant exiting refrigeration compressor 64 aftercooler is cooled down to cryogenic levels first in the NRU second heat exchanger 65 and then the NRU distillation column reboiler 68. The condensed HP refrigerant then enters refrigerant vessel 70. Liquid from refrigerant vessel 70 is split into two streams. The first HP refrigerant stream is directed via conduit 74 to be subcooled in NRU third heat exchanger 66. This stream is then flashed across an expansion valve 101 to become medium pressure (MP) refrigerant supply to side condenser 48 via conduit 76. Conduit 76 then directs the MP refrigerant to be warmed in NRU third heat exchanger 66 and then NRU second heat exchanger 65, and the stream is then returned to an intermediate stage of refrigeration compressor 62 at ambient temperature. The other portion of the HP refrigerant liquid leaving refrigerant vessel 70 is directed via conduit 78 to be subcooled in NRU second heat exchanger 65. This portion of the HP refrigerant is then flashed across an expansion valve 103 to become the low pressure (LP) refrigerant supply to NRU distillation column overhead condenser 49 via conduit 80. Conduit 80 then directs the LP refrigerant to be warmed to ambient temperature in NRU second heat exchanger 65 and then to the low stage suction of refrigeration compressor 62. The final stage discharge of refrigeration compressor 64 includes the total refrigerant stream and completes the refrigeration loop. In this example, conduit 82 provides a refrigerant makeup stream which may be supplied from the NRU distillation column 26 on an intermittent basis (using manual, semi-automatic, or automatic control) via valve 105, and conduit 84 and valve 107 allow an operator (using manual, semi-automatic, or automatic control) to intermittently blowdown excess refrigerant, returning the refrigerant to NRU distillation column 26.

The NRU heat exchangers 12, 48, 49, 65, 66, and 68 and refrigerant vessel 70 in embodiments 300, 400, and 500 as illustrated schematically in FIGS. 2, 3, and 4 represent a typical configuration of the heat exchanger network and refrigeration loop within an NRU. However, other heat exchanger network configurations may be used in the NRU. For example, the first heat exchanger 12 may be combined with third heat exchanger 66 into one unit; or alternatively first heat exchanger 12 may be combined with the NRU column reboiler 68 into one unit. Other combinations of stream pairings in various multi-stream heat exchangers may make thermodynamic sense and are possible. Alternate locations for the refrigerant vessel 70 or additional refrigerant vessels may also be used in the NRU. The optimum configuration of the heat exchanger network in the NRU and number of refrigerant vessels will depend on the specific system design parameters. Additional streams not illustrated in embodiments 300, 400, and 500 may also be present in the heat exchanger network, when additional heat integration is employed or when additional product streams are required.

Importantly, embodiments 300, 400, and 500 differ in the details of PMSS 8 in order that demethanizer column 28 may operate at a pressure lower than, equal to, or just above the NRU distillation column 26 pressure. In embodiment 300, PMSS 8 features the addition of a conduit 10 allowing demethanizer column 28 overhead to be routed to NRU first heat exchanger 12, a separator vessel 14, and conduit 18 to route condensed demethanizer overhead (NRU feed) to a pump 16. The addition of separator vessel 14 and pump 16 to NRU 6, as well as conduits 10, 18 and 19, allow for demethanizer column 28 to operate at a pressure below, equal to, or just above that of NRU distillation column 26. NRU distillation column bottoms stream, which is low in nitrogen, is routed through bottoms conduit 20 so that it may be partially revaporized and cooled by passing through expansion valve 22, and conduit 24 routes the expanded stream through NRU first heat exchanger 12 and then conduit 25 routes the stream back to the NGL recovery unit 4 (the gas subcooled process in this example) as a cold stream for subcooler (heat exchanger) 30. Conduit 19 allows any uncondensed demethanizer overhead from separator 14 to bypass the NRU distillation column 26 by blending the stream with the expanded NRU distillation column bottoms upstream of NRU first heat exchanger 12. In sum, as illustrated schematically in FIG. 2, embodiment 300 feeds the overhead from demethanizer column 28 to cross exchange in NRU first heat exchanger 12, then to separator vessel 14 and pump 16. The liquid separated out in separator vessel 14 is fed to pump 16 for feeding into NRU distillation column 26 of NRU 6. The bottoms from NRU distillation column 26 is expanded across an expansion valve and fed back to join with any uncondensed demethanizer overhead vapor from separator vessel 14 to subcooler 30 of NGL recovery unit 4 after heating in NRU first heat exchanger 12. In embodiment 300, without pump 16 and separator vessel 14, demethanizer column 28 would have to operate at a pressure that is higher than NRU distillation column 26. The result would be higher overall compressor horsepower consumption, when considering the aggregate of the NGL recovery unit 4 residue compressor and refrigeration compressor power as well as the NRU 6 refrigeration compressor power.

Process conditions and overall material balance for an example of Embodiment 300 illustrated schematically in FIG. 2 are presented in Tables 2 and 3, however, these conditions and flow rates are to be considered representative and actual conditions and flows may vary depending upon design parameters.

TABLE 2
Example Process Conditions - Embodiment 300

| | Pressure | Pressure | Temp. | Temp. |
|---|---|---|---|---|
| Description | (psia) | (kPa) | (° F.) | (C.) |
| NGL Plant Inlet (34) | 914 | 6302 | 120 | 49 |
| Cold Separator (40) | 899 | 6198 | −15 | −26 |
| Demethanizer Overhead (10) | 301 | 2075 | −147 | −99 |
| Demethanizer Bottoms (32) | 305 | 2103 | 62 | 17 |
| Residue Product (36) | 269 | 1855 | 153 | 67 |
| PMSS Separator (14) | 291 | 2006 | −171 | −113 |
| NRU Column Feed (21) | 379 | 2613 | −169 | −112 |
| NRU Column Overhead (48) | 368 | 2537 | −243 | −153 |
| Nitrogen Reject Stream (52) | 363 | 2503 | 110 | 43 |
| NRU Column Bottoms (20) | 374 | 2579 | −149 | −101 |
| Flashed NRU Column Bottoms | 230 | 1586 | −172 | −113 |
| (24) | | | | |
| CH₄ Product from NRU (25) | 228 | 1572 | −162 | −108 |

TABLE 3
Example Overall Material Balance - Embodiment 300

| | Mol % | Mol % | Mol % | Mol % | Total |
|---|---|---|---|---|---|
| | N2 | CH4 | C2 | C3+ | lbmol/hr |
| NGL Plant Feed | 5.00 | 74.27 | 12.15 | 8.58 | 21,959,81 |
| (34) | | | | | |
| NGL Product (32) | 0.00 | 0.83 | 56.21 | 42.96 | 4,368.96 |
| Residue (36) | 2.94 | 95.77 | 1.25 | 0.04 | 16,991.38 |
| Reject Nitrogen | 99.90 | 0.10 | 0.00 | 0.00 | 599.47 |
| (52) | | | | | |

In certain alternative embodiments of the methods and systems of the present disclosure, a different natural gas recovery process may be used if the process includes a demethanizer or deethanizer that operates at temperatures below ambient.

Embodiment 400 differs from embodiment 300 by the following features. A separator vessel 54 is positioned at an outlet of NGL recovery unit expander 42, allowing an expander outlet vapor portion (separated out by separator vessel 54) to be routed through a conduit 43 to NRU first heat exchanger 12 then via conduit 21 to feed NRU 6 at near bottom location 50 of NRU distillation column 26. NRU distillation column 26 bottoms, with the reduced nitrogen content, is expanded across expansion valve 22 via conduit 20 and reheated in NRU first heat exchanger 12 via conduit 24 and is returned to NGL recovery unit 4 and fed via conduit 60 to demethanizer column 28 at middle feed location 46 where expander 42 outlet normally feeds demethanizer column 28. An expander outlet liquid stream (separated out by separator vessel 54) is routed through a conduit 58 and also fed to middle feed location in demethanizer 28 by joining with the expanded NRU distillation column bottoms at a point in conduit 60, bypassing NRU 6 altogether. In the integrated NGL recovery unit/NRU disclosed herein for embodiment 400, the expander outlet pressure may be higher than the normal expander outlet pressure of an NGL recovery unit that is not operatively connected to an NRU. The combined middle feed in conduit 60 is returned to demethanizer column 28 at similar conditions to a demethanizer column 28 “idle feed” with no NRU, allowing NGL recovery unit 4 to operate very closely to operations with no NRU. However, the middle feed to demethanizer column 28 is reduced in nitrogen such that the demethanizer column 28 overhead residue has a nitrogen content that meets pipeline specifications. In sum, expander 42 feeds separator vessel 54 which is upstream of demethanizer column 28. Conduit 56 routes separator vessel 54 vapors to NRU distillation column 26 after further cooling in the NRU, and conduit 58 routes separator vessel 54 bottoms to demethanizer column 28. Bottoms from NRU distillation column 26 are fed back to demethanizer column 28 after expansion and reheating in the NRU, where the stream joins with the liquid from separator vessel 54, entering demethanizer column 28 as the middle feed.

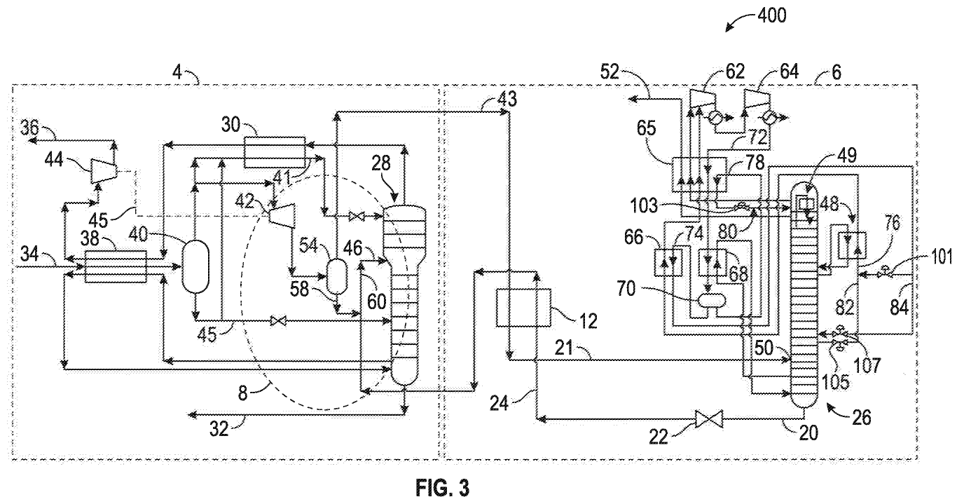

Process conditions and overall material balance for embodiment 400 illustrated schematically in FIG. 3 are presented in Tables 4 and 5, however, these conditions and flow rates are to be considered representative and actual conditions and flows can vary depending upon design parameters.

TABLE 4
Example Process Conditions - Embodiment 400

| | Pressure | Pressure | Temp. | Temp. |
|---|---|---|---|---|
| Description | (psia) | (kPa) | (° F.) | (C.) |
| NGL Plant Inlet (34) | 914 | 6302 | 120 | 49 |
| Cold Separator (40) | 899 | 6198 | −14 | −26 |
| Demethanizer Overhead (10) | 250 | 1724 | −146 | −99 |
| Demethanizer Bottoms (32) | 254 | 1751 | 47 | 8.3 |
| Residue Product (36) | 287 | 1979 | 146 | 63 |
| Expander Outlet (43) | 379 | 2613 | −78 | −61 |
| NRU Column Feed (21) | 374 | 2579 | −151 | −102 |
| NRU Column Overhead (49) | 368 | 2537 | −243 | −153 |
| Nitrogen Reject Stream (52) | 363 | 2503 | 110 | 43 |
| NRU Column Bottoms (20) | 374 | 2579 | −146 | −99 |
| Flashed NRU Column Bottoms | 259 | 1786 | −163 | −108 |
| (24) | | | | |
| Demethanizer Middle Feed (60) | 254 | 1751 | −97 | −72 |

TABLE 5
Example Overall Material Balance - Embodiment 400

| | Mol % | Mol % | Mol % | Mol % | Total |
|---|---|---|---|---|---|
| | N2 | CH4 | C2 | C3+ | lbmol/hr |
| NGL Plant Feed (34) | 5.00 | 74.27 | 12.15 | 8.58 | 21,959.81 |
| NGL Product (32) | 0.00 | 0.83 | 55.12 | 44.05 | 4,260.19 |
| Residue (36) | 2.95 | 95.14 | 1.87 | 0.04 | 17,105.71 |
| Reject Nitrogen (52) | 99.90 | 0.10 | 0.00 | 0.00 | 593.91 |

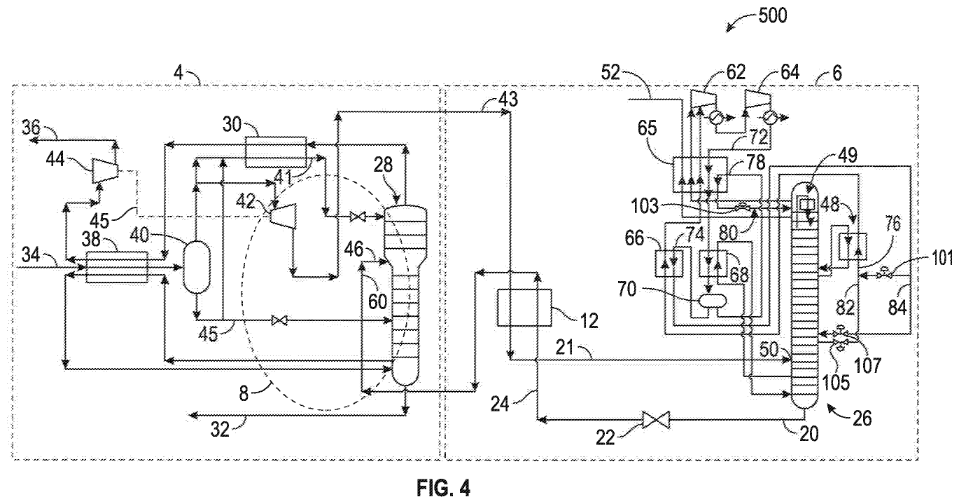

Embodiment 500 differs from embodiments 300 and 400 by the following features. As illustrated schematically in FIG. 4, embodiment 500 is similar to embodiment 400; the outlet from expander 42 is routed via conduit 43 to NRU first heat exchanger 12 then via conduit 21 to feed NRU distillation column 26 at location 50, while the reduced nitrogen bottom liquid from NRU distillation column 26 is expanded across expansion valve 22 via conduit 20 and reheated in NRU first heat exchanger 12 via conduit 24 and routed to demethanizer column 28 middle feed via conduit 60. However, in embodiment 500, FIG. 4, there is no separator vessel between expander 42 and demethanizer 28. Instead, both the liquid and vapor outlet of expander 42 feeds NRU 6 directly via conduit 43. In the integrated NGL recovery unit/NRU disclosed herein as embodiment 500, the expander outlet pressure may be higher than the normal expander outlet pressure of an NGL recovery unit that is not operatively connected to an NRU. In this manner, demethanizer column 28 may be operated at a lower, equivalent, or slightly higher pressure than NRU distillation column 26.

Process conditions and overall material balance for embodiment 500 illustrated schematically in FIG. 4 are presented in Tables 6 and 7, however, these conditions and flow rates are to be considered representative and actual conditions and flows can vary depending upon design parameters.

TABLE 6
Example Process Conditions - Embodiment 500

| | Pressure | Preesure | Temp. | Temp. |
|---|---|---|---|---|
| Description | (psia) | (kPa) | (° F.) | (C.) |
| NGL Plant Inlet (34) | 914 | 6302 | 120 | 49 |
| Cold Separator (40) | 899 | 6198 | −14 | −26 |
| Demethanizer Overhead | 250 | 1724 | −146 | −99 |
| Demethanizer Bottoms (32) | 254 | 1751 | 47 | 8.3 |
| Residue Product (36) | 287 | 1979 | 146 | 63 |
| Expander Outlet (43) | 379 | 2613 | −78 | −61 |
| NRU Column Feed (21) | 374 | 2579 | −147 | −99 |
| NRU Column Overhead (49) | 368 | 2537 | −243 | −153 |
| Nitrogen Reject Stream (52) | 363 | 2503 | 110 | 43 |
| NRU Column Bottoms (20) | 374 | 2579 | −142 | −97 |
| Flashed NRU Column Bottoms | 259 | 1786 | −160 | −107 |
| (24) | | | | |
| Demethanizer Middle Feed (60) | 254 | 1751 | −97 | −72 |

TABLE 7
Example Overall Material Balance - Embodiment 500

| | Mol % | Mol % | Mol % | Mol % | Total |
|---|---|---|---|---|---|
| | N2 | CH4 | C2 | C3+ | lbmol/hr |
| NGL Plant Feed (34) | 5.00 | 74.27 | 12.15 | 8.58 | 21,959.81 |
| NGL Product (32) | 0.00 | 0.83 | 55.12 | 44.05 | 4,260.21 |
| Residue (36) | 2.93 | 95.16 | 1.87 | 0.04 | 17,101.51 |
| Reject Nitrogen (52) | 99.90 | 0.10 | 0.00 | 0.00 | 598.09 |

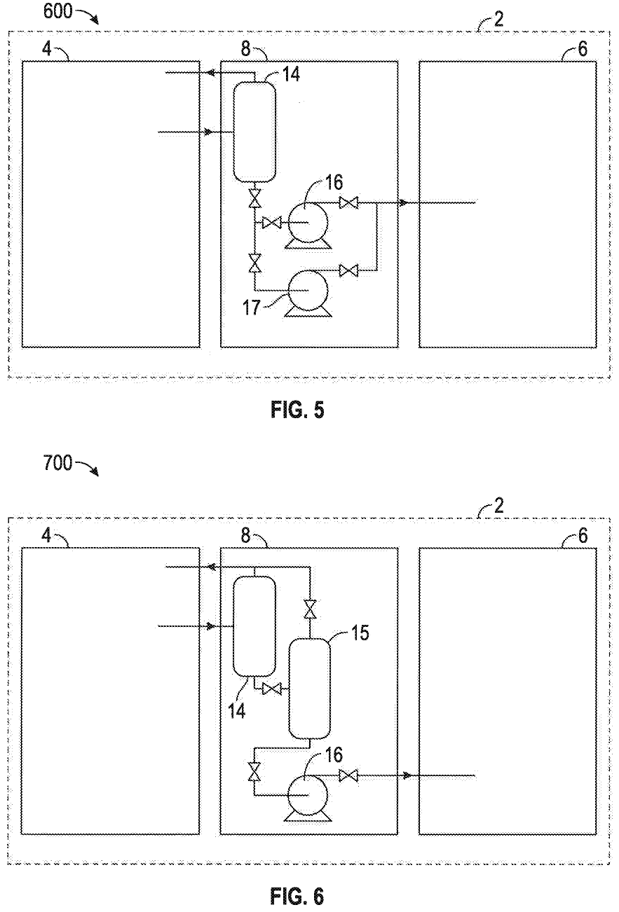

FIGS. 5 and 6 are highly schematic illustrations of alternative system embodiments 600 and 700, respectively, in accordance with the present disclosure. Embodiment 600 includes redundancy in the form of two PMSS pumps (16, 17) connected in parallel, each fluidly connected to separator vessel 14. Embodiment 600 allows NGL recovery unit 4 and NRU 6 to be used with pump 16 or pump 17, or both, through use of suitable isolation valves (as illustrated but not referenced). Alternatively, PMSS pumps 16, 17 may be configured with suitable valving and piping so that they may be used in series or parallel flow arrangement. Alternative embodiments may be considered with three or more pumps connected in either parallel or series configuration, or a combination of the two.

Embodiment 700 includes two separator vessels (14, 15) serving a single pump 16. Embodiment 700 allows NGL recovery unit 4 and NRU 6 to be used with an additional separator expansion or flash stage. Separator vessels 14 and 15 may operate in conjunction with each other, for example separator vessel 14 at a relatively high to moderate pressure, while separator vessel 15 operates at a moderate to low pressure. Alternatively, separator vessels 14, 15 may be configured with suitable valving and piping so that they may be used in series or parallel flow arrangement. Alternative embodiments may be considered where separator vessels 14 and 15 feed two or more pumps, in series and/or parallel configuration, and the pumps may be arranged to be common to separators 14 and 15 or, alternatively, one or more pumps may be dedicated to separator 14 and one or more pumps may be dedicated to separator 15.

Any known type of NGL recovery unit and NRU may be employed in practicing the methods and systems of the present disclosure. Suitable NGL recovery units and components typically used therewith include those described in U.S. Pat. Nos. 4,157,904; 4,617,039; 4,718,927; 4,895,584; 5,771,712; 5,799,507; 6,182,469; 6,278,035; 6,311,516; 7,544,272; 9,487,458; and 9,726,426; and NRUs discussed in U.S. Pat. Nos. 5,141,544; 5,257,505; 5,375,422; 8,794,031; 9,003,829; 9,816,752; 9,487,458; and 9,726,426.

Any known type of mass flow meter may be employed in practicing the methods and systems of the present disclosure. Suitable mass flow meters and components typically used therewith include the coriolis flow and density meters currently commercially available from Emerson (under the trade designation ELITE Peak Performance Coriolis Flow and Density Meter) and other suppliers. Any known type of pressure relief component (PRV, burst disc, or other) may be employed in practicing the methods and systems of the present disclosure. Suitable pressure relief components include those currently commercially available from Anderson Greenwood (USA) or from Expro, London (U.K.) under the trade designation PRV MAX. Any known type of expansion valve may be employed in practicing the methods and systems of the present disclosure, including those currently commercially available from Samson Controls Inc. USA. Suitable separators include those commercially available from ASME Section VIII coded pressure vessel manufacturers. Any known type of cryogenic pump may be employed in practicing the methods and systems of the present disclosure, including positive displacement, centrifugal, horizontal, vertical pumps, and pumps operated with variable speed motors. Suitable pumps include those currently available from CryoStar (France) or Nikkiso (Japan). Suitable conduits and components typically used therewith include currently commercially available pipe from Hydrocarbon Processing Industry (HPI) manufacturers such as Tenaris (Luxumbourg).

During certain methods of the present disclosure, one or all of T, P, mass flow rate, gas or vapor concentrations (or percentages of set point values) inside and/or outside the pressure management sub-system(s) may be displayed locally on one or more Human Machine Interfaces (HMI), such as a laptop computer having a display screen having a graphical user interface (GUI), or handheld device, or similar, either inside or outside (or both) of pressure management sub-system. In certain embodiments the HMI may record and/or transmit the data via wired or wireless communication to another HMI, such as another laptop, desktop, or hand-held computer or display. These communication links may be wired or wireless.

The NGL recovery unit, NRU, and PMSS may be made of metals, except where rubber or other polymeric seals may be employed. Suitable metals include stainless steels, for example, but not limited to, 306, 316, as well as titanium alloys, aluminum alloys, and the like. High-strength materials like C-110 and C-125 metallurgies that are NACE qualified may be employed. (As used herein, “NACE” refers to the corrosion prevention organization formerly known as the National Association of Corrosion Engineers, now operating under the name NACE International, Houston, Tex.) Use of high strength steel and other high strength materials may significantly reduce the wall thickness required, reducing weight. Threaded connections may eliminate the need for 3^{rd} party forgings and expensive welding processes—considerably improving system delivery time and overall cost. It will be understood, however, that the use of 3^{rd} party forgings and welding is not ruled out for system components described herein and may actually be preferable in certain situations. The skilled artisan, having knowledge of the particular application, pressures, temperatures, and available materials, will be able design the most cost effective, safe, and operable system components for each particular application without undue experimentation.

One or more control strategies may be employed, as long as the strategy includes measurement of NGL recovery unit demethanizer column pressure and NRU distillation column pressure, as well as measurements to be able to determine product purities and flow rates achieved, and those measurements (or values derived from those measurements) may be used in controlling the systems and/or processes described herein. A pressure process control scheme may be employed, for example in conjunction with the pressure control devices and mass flow controllers. A master controller may be employed, but the disclosure is not so limited, as any combination of controllers may be used. Programmable logic controllers (PLCs) may be used.

Control strategies may be selected from proportional-integral (PI), proportional-integral-derivative (PID) (including any known or reasonably foreseeable variations of these), and may compute a residual equal to a difference between a measured value and a set point to produce an output to one or more control elements. The controller may compute the residual continuously or non-continuously. Other possible implementations of the disclosure are those wherein the controller comprises more specialized control strategies, such as strategies selected from feed forward, cascade control, internal feedback loops, model predictive control, neural networks, and Kalman filtering techniques.

FIGS. 7A and 7B; 8A and 8B; and 9A and 9B are schematic logic diagrams of three method embodiments, where embodiment 800 comprises routing one or more raw natural gas streams to a natural gas processing plant, the natural gas processing plant comprising an NGL recovery unit including a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent, Box 802.

Method embodiment 800 further comprises removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a reject nitrogen stream and the product natural gas, Box 804.

Method embodiment 800 further comprises (Box 806) operating the demethanizer column at a pressure lower than, equal to, or just above the NRU distillation column by managing a pressure relationship between the demethanizer column and the NRU distillation column using a PMSS comprising a separator, a pump and an expansion valve, comprising:
(i) routing at least a portion of demethanizer column overhead to one or more heat exchangers within the NRU to partially or wholly condense the demethanizer overhead feeding the one or more heat exchangers;
(ii) routing the partially or wholly condensed demethanizer overhead to a separator to separate any remaining vapor from the liquid;
(iii) pumping the liquid from the separator into a lower section of the NRU distillation column using the pump;
(iv) combining any uncondensed demethanizer overhead vapor leaving the separator with the low nitrogen NRU distillation column bottoms stream after the NRU distillation column bottoms has been reduced in pressure via an expansion valve, whereby the combined stream can now be called the natural gas product;
(v) routing the natural gas product to one or more heat exchangers within the NRU to reheat the stream to a temperature that is similar to the demethanizer overhead temperature; and
(vi) routing the natural gas product leaving the NRU to one or more heat exchangers within the NGL recovery unit to heat the natural gas product to ambient temperature.

Method embodiment 900, illustrated schematically in FIGS. 8A and 8B, comprises routing one or more raw natural gas streams to a natural gas processing plant, the natural gas processing plant comprising an NGL recovery unit including an expander upstream of a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent, Box 902.

Method embodiment 900 further comprises removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a reject nitrogen stream and the product natural gas, Box 904.

Method embodiment 900 further comprises (Box 906) operating the demethanizer column at a pressure lower than, equal to, or just above the NRU distillation column by managing a pressure relationship between the demethanizer column and the NRU distillation column using a PMSS comprising a separator and an expansion valve, comprising:
(i) routing an expander outlet stream to a separator, forming a separator substantially vapor stream and a separator substantially liquid stream;
(ii) routing the separator substantially vapor stream through one or more heat exchangers within the NRU and into a lower section of the NRU distillation column, the NRU distillation column removing a major portion of the nitrogen contained in the separator substantially vapor stream to form a low nitrogen NRU distillation column bottoms;
(iii) routing the low nitrogen NRU distillation column bottoms to an expansion valve, forming an expanded low nitrogen NRU distillation column bottoms;
(iv) routing the expanded low nitrogen NRU distillation column bottoms through the one or more heat exchangers; and
(v) combining the expanded low nitrogen expanded NRU distillation column bottoms leaving the one or more heat exchangers with the separator substantially liquid stream, forming a demethanizer column feed stream that feeds the demethanizer column at a same or similar middle location of a comparable demethanizer column having no operatively connected NRU, the demethanizer column forming a demethanizer column overhead and a demethanizer column bottoms, both of which are routed out of the natural gas processing plant without going through the NRU distillation column.

Method embodiment 950, illustrated schematically in FIGS. 9A and 9B, comprises routing one or more raw natural gas streams to a natural gas processing plant, the natural gas processing plant comprising an NGL recovery unit including an expander upstream of a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent, Box 952.

Method embodiment 950 further comprises removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a reject nitrogen stream and the product natural gas, Box 954.

Method embodiment 950 further comprises (Box 956) operating the demethanizer column at a pressure lower than, equal to, or just above the NRU distillation column by managing a pressure relationship between the demethanizer column and the NRU distillation column using a PMSS comprising an expansion valve but no separator, comprising:
(i) routing an expander outlet liquid and vapor through one or more heat exchangers within the NRU and into a lower section of the NRU distillation column so that a major portion of the nitrogen contained in the stream can be removed, forming a low nitrogen NRU distillation column bottoms;
(ii) routing the low nitrogen NRU distillation column bottoms to an expansion valve, forming an expanded low nitrogen NRU distillation column bottoms;
(iii) routing the expanded low nitrogen NRU distillation column bottoms through the one or more heat exchangers; and
(iv) routing the expanded low nitrogen NRU distillation column bottoms leaving the one or more heat exchangers to feed the demethanizer at the same or similar middle feed location of a comparable demethanizer column having no operatively connected NRU, the demethanizer column forming a demethanizer column overhead and a demethanizer column bottoms, both of which are routed out of the natural gas processing plant without going through the NRU distillation column.

Pressure management sub-systems may be built to meet ISO standards, Det Norske Veritas (DNV) standards, American Bureau of Standards (ABS) standards, American Petroleum Institute (API) standards, and/or other standards. It may be possible to route a raw natural gas stream to an NRU first, to form a nitrogen reject stream and a reduced nitrogen natural gas stream, and then route the reduced nitrogen natural gas stream to an NGL recovery unit in order to form the natural gas product and an NGL product stream; however, in such embodiments the PMSS may require a completely different arrangement of one or more pumps, separators, and conduits than explained herein.

The electrical connections, if used (voltage and amperage) will be appropriate for the zone rating desired of each system. In certain embodiments one or more electrical cables may be run and connected to an identified power supply at the work site to operate the HMI, NGL recovery unit, NRU, and PMSS. Certain embodiments may employ a dedicated power supply. The identified or dedicated power supply may be controlled by one or more logic devices so that it may be shut down. In exemplary embodiments, systems of the present disclosure may have an electrical isolation (lockout) device on a secure cabinet.

In embodiments where connection to one or more remote HMI units is desired, this may be achieved by an intrinsically safe cable and connection to allow system components to operate in the required zoned area. If no remote access is required, power to operate the HMI, NGL recovery unit, NRU, and PMSS may be integral to the apparatus, such as batteries, for example, but not limited to, Li-ion batteries. In these embodiments, the power source may be enclosed allowing it to operate in a zoned area (Zone 0 (gases) in accordance with International Electrotechnical Commission (IEC) processes). By “intrinsically safe” is meant the definition of intrinsic safety used in the relevant IEC apparatus standard IEC 60079-11, defined as a type of protection based on the restriction of electrical energy within apparatus and of interconnecting wiring exposed to a potentially explosive atmosphere to a level below that which can cause ignition by either sparking or heating effects. For more discussion, see “AN9003—A User's Guide to Intrinsic Safety”, retrieved from the Internet Jul. 12, 2017, and incorporated herein by reference.

In certain embodiments, internal algorithms in the logic device, such as a PLC, may calculate a rate of increase or decrease in pressure inside the PMSS and/or the NGL recovery unit, and/or the NRU. This may then be displayed or audioed in a series of ways such as “percentage to shutdown” lights or sounds, and the like on one or more GUIs. In certain embodiments, an additional function within an HMI may be to audibly alarm when the calculated pressure rate of increase or decrease reaches a level set by the operator. In certain embodiments this alarm may be sounded inside the pressure management sub-system, outside the pressure management sub-system, as well as remote from the pressure management sub-system, for example in a local or remote control room.

Pressure management sub-systems, conduits therefore, separators, pumps, logic devices, sensors, expansion and non-expansion valves, and optional safety shutdown units should be capable of withstanding long term exposure to probable liquids and vapors, including hydrocarbons, acids, acid gases, fluids (oil-based and water-based), solvents, brine, anti-freeze compositions, hydrate inhibition chemicals, and the like, typically encountered in hydrocarbon processing facilities and cryogenic processing facilities.

In alternative embodiments, the pressure management sub-system may be enclosed within a frame or cabinet, and/or truck-mounted, and/or ship-mounted. Moreover, the various components (such as separators) need not have specific shapes or specific conduit routing as illustrated in the drawings, but rather the pressure management sub-system separators could take any shape, such as a box or cube shape, elliptical, triangular, prism-shaped, hemispherical or semi-hemispherical-shaped (dome-shaped), or combination thereof and the like, as long as the separator performs the desired separation. The conduit and column cross-sections need not be round, but may be rectangular, triangular, round, oval, and the like. It will be understood that such embodiments are part of this disclosure and deemed with in the claims. Furthermore, one or more of the various components may be ornamented with various ornamentation produced in various ways (for example stamping or engraving, or raised features such as reflectors, reflective tape), such as facility designs, operating company designs, logos, letters, words, nicknames (for example LINDE, and the like). Components of the NGL recovery unit, NRU and/or PMSS may include optional hand-holds, which may be machined or formed to have easy-to-grasp features for fingers, or may have rubber grips shaped and adorned with ornamental features, such as raised knobby gripper patterns.

Thus the methods and systems described herein afford ways to perform natural gas recovery and nitrogen rejection therefrom safely and economically.

Embodiments disclosed herein include:

A: A method comprising (or consisting essentially of, or consisting of):

(a) routing one or more raw natural gas streams to a natural gas processing plant, the natural gas processing plant comprising an NGL recovery unit including a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent;

(b) removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a nitrogen reject stream and the product natural gas; and

(c) operating the demethanizer column at a pressure lower than, equal to, or just above the NRU distillation column by managing a pressure relationship between the demethanizer column and the NRU distillation column using a PMSS comprising a separator, a pump, and an expansion valve, comprising:
(i) routing at least a portion of demethanizer column overhead to one or more heat exchangers within the NRU to partially or wholly condense the demethanizer overhead feeding the one or more heat exchangers;
(ii) routing the partially or wholly condensed demethanizer overhead to a separator to separate any remaining vapor from the liquid;
(iii) pumping the liquid from the separator into a lower section of the NRU distillation column using the pump;
(iv) combining any uncondensed demethanizer overhead vapor leaving the separator with the NRU distillation column bottoms stream after the NRU distillation column bottoms has been reduced in pressure via an expansion valve, whereby the combined stream can now be called the natural gas product;
(v) routing the natural gas product to one or more heat exchangers within the NRU to reheat the stream to a temperature that is similar to the demethanizer overhead temperature; and
(vi) routing the natural gas product leaving the NRU to one or more heat exchangers within the NGL recovery unit to heat the natural gas product to ambient temperature.

B: A method comprising (or consisting essentially of, or consisting of):

(a) routing one or more raw natural gas streams to a natural gas processing plant, the natural gas processing plant comprising an NGL recovery unit including an expander upstream of a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent;

(b) removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a reject nitrogen stream and the product natural gas; and

(c) operating the demethanizer column at a pressure lower than, equal to, or just above the NRU distillation column by managing a pressure relationship between the demethanizer column and the NRU distillation column using a PMSS comprising a separator and an expansion valve, comprising:
(i) routing an expander outlet stream to a separator, forming a separator substantially vapor stream and a separator substantially liquid stream;
(ii) routing the separator substantially vapor stream through one or more heat exchangers within the NRU and into a lower section of the NRU distillation column, the NRU distillation column removing a major portion of the nitrogen contained in the separator substantially vapor stream to form a low nitrogen NRU distillation column bottoms;
(iii) routing the low nitrogen NRU distillation column bottoms to an expansion valve, forming an expanded low nitrogen NRU distillation column bottoms;
(iv) routing the expanded low nitrogen NRU distillation column bottoms through the one or more heat exchangers, forming a cooled and expanded low nitrogen NRU distillation column bottoms; and
(v) combining the expanded low nitrogen NRU distillation column bottoms leaving the one or more heat exchangers with the separator substantially liquid stream, forming a demethanizer column feed stream that feeds the demethanizer column at a same or similar middle location of a comparable demethanizer column having no operatively connected NRU, the demethanizer column forming a demethanizer column overhead and a demethanizer column bottoms, both of which are routed out of the natural gas processing plant without going through the NRU distillation column.

C: A method comprising (or consisting essentially of, or consisting of):

(a) routing one or more raw natural gas streams to a natural gas processing plant, the natural gas processing plant comprising an NGL recovery unit including an expander upstream of a demethanizer column, and an NRU including a distillation column, the one or more raw natural gas streams comprising (or consisting essentially of, or consisting of) methane, C₂+ hydrocarbons, and nitrogen, the nitrogen having a concentration greater than about 3 mole percent, or greater than about 4 mole percent;

(b) removing a majority of the C₂+ hydrocarbons using the NGL recovery unit and a sufficient amount of the nitrogen using the NRU to form a product natural gas, wherein the removing step comprises removing the majority of the C₂+ hydrocarbons from the raw natural gas to form an NGL stream rich in C₂+ hydrocarbons and a residue stream, followed by removing the sufficient amount of the nitrogen from the residue stream to form a reject nitrogen stream and the product natural gas; and

(c) operating the demethanizer column at a pressure lower than, equal to, or just above the NRU distillation column by managing a pressure relationship between the demethanizer column and the NRU distillation column using a PMSS comprising an expansion valve but no separator, comprising:
(i) routing an expander outlet liquid and vapor through one or more heat exchangers within the NRU and into a lower section of the NRU distillation column so that a major portion of the nitrogen contained in the stream can be removed, forming a low nitrogen NRU distillation column bottoms;
(ii) routing the low nitrogen NRU distillation column bottoms to an expansion valve, forming an expanded low nitrogen NRU distillation column bottoms;
(iii) routing the expanded, low nitrogen NRU distillation column bottoms through the one or more heat exchangers; and
(iv) routing the expanded, low nitrogen NRU distillation column bottoms leaving the one or more heat exchangers to feed the demethanizer at the same or similar middle feed location of a comparable demethanizer column having no operatively connected NRU, the demethanizer column forming a demethanizer column overhead and a demethanizer column bottoms, both of which are routed out of the natural gas processing plant without going through the NRU distillation column.

D: A system comprising (or consisting essentially of, or consisting of):

(a) an NGL recovery unit including a demethanizer column;

(b) an NRU including a distillation column; and

(c) a pressure management sub-system (PMSS) operatively and fluidly connected to the NGL recovery unit and the NRU, the PMSS comprising at least first through seventh conduits (inclusive), a separator, a pump, and an expansion valve, and further comprising:
(i) the first conduit configured to route at least a portion of demethanizer column overhead to one or more heat exchangers and then to the separator;
(ii) the second conduit to route a liquid stream from the separator to a pump;
(iii) the third conduit to route the liquid from the separator into a lower section of the NRU distillation column using the pump;
(iv) the fourth conduit configured to route at least a portion of reduced nitrogen NRU distillation column bottoms to the expansion valve and the fifth conduit to route this portion of the NRU distillation column bottoms to the heat exchanger or exchangers;
(v) the sixth conduit to blend any vapor leaving the separator with the NRU distillation column bottoms downstream of the expansion valve and upstream of the heat exchanger or exchangers to form the natural gas product; and
(vi) the seventh conduit to route the natural gas product leaving the one or more heat exchangers to the NGL recovery unit heat exchanger network.

E: A system comprising (or consisting essentially of, or consisting of):

(a) an NGL recovery unit including a demethanizer column;

(b) an NRU including an NRU distillation column; and

(c) a pressure management sub-system (PMSS) operatively and fluidly connected to the NGL recovery unit and the NRU, the PMSS comprising first through ninth conduits
(inclusive), a separator, and an expansion valve, and further comprising:
(i) the first conduit configured to route an expander outlet stream to a separator, forming a separator substantially vapor stream and a separator substantially liquid stream;
(ii) the second conduit configured to route the separator substantially vapor stream from the separator through one or more heat exchangers and then to the third conduit to route the stream into a lower section of the NRU distillation column;
(iii) the fourth conduit configured to route reduced nitrogen NRU distillation column bottoms to an expansion valve, forming the expanded NRU distillation column bottoms;
(iv) the fifth conduit configured to route expanded NRU distillation column bottoms through one or more heat exchangers prior to combining with the separator substantially liquid stream; and
(v) the sixth conduit configured to combine the separator substantially liquid stream with the expanded NRU distillation column bottoms leaving the heat exchanger or exchangers, to form a demethanizer feed stream which flows through the seventh conduit configured to route the demethanizer feed stream to a middle feed location of the demethanizer column, and the eighth conduit configured to route demethanizer column overhead and the ninth conduit configured to route demethanizer column bottoms, where the eighth and ninth conduits route the streams out of the natural gas processing plant without going through the NRU distillation column.

F: A system comprising (or consisting essentially of, or consisting of):

(a) an NGL recovery unit including a demethanizer column;

(b) an NRU including a distillation column; and

(c) a pressure management sub-system (PMSS) operatively and fluidly connected to the NGL recovery unit and the NRU, the PMSS comprising at least first through seventh conduits (inclusive) and an expansion valve, and further comprising:
(i) the first conduit configured to route an expander outlet stream through one or more heat exchangers and then to the second conduit to route the stream into a lower section of the NRU distillation column;
(ii) the third conduit configured to route reduced nitrogen NRU distillation column bottoms to an expansion valve, forming an expanded NRU distillation column bottoms;
(iii) the fourth conduit configured to route the expanded NRU distillation column bottoms through one or more heat exchangers;
(iv) the fifth conduit route the expanded NRU distillation column bottoms leaving the heat exchanger or exchangers to feed a middle location of the demethanizer column, and the sixth conduit configured to route demethanizer column overhead and the seventh conduit configured to route demethanizer column bottoms, where the sixth and seventh conduits route the streams out of the natural gas processing plant without going through the NRU distillation column.

Each of the embodiments A, B, C, D, E, and F may have one or more of the following additional elements in any combination:

Element 1. Methods and systems wherein the raw natural gas stream may be routed to the NGL recovery unit prior to the NRU.

Element 2. Methods and systems wherein the raw natural gas stream may be routed to the NRU prior to the NGL recovery unit.

Element 3. Methods and systems wherein the NGL recovery unit may comprise a gas-subcooled or related process, wherein the raw natural gas may be routed through one or more heat exchangers to produce one or more sub-cooled raw natural gas feed streams to the demethanizer column.

Element 4: Methods and systems wherein the PMSS may comprise one or more redundant components, for example, two or more expansion valves arranged in parallel flow relationship, or two or more pumps arranged in parallel flow relationship, or two or more separators arranged in parallel flow relationship.

Element 5: Methods and systems wherein the PMSS may be arranged in series flow relationship, for example, two or more separators arranged in series, where liquid separated from upstream separators is caused to flow into a downstream separator.

Element 6: Methods and systems with mixed parallel and series flow are also contemplated, for example, an arrangement of four separators where first and second separators are arranged in parallel with each other, third and fourth separators are arranged in parallel with each other, and where the first is in series with the third, and the second is in series with the fourth.

Element 7: Methods and systems wherein the separator and pump are sized sufficiently so that the demethanizer column operates at a pressure lower than, equal to, or just above the NRU distillation column.

Element 8: Methods and systems wherein the NGL recovery unit includes at least one raw natural gas cooling heat exchanger and at least one separator for forming at least one sub-cooled raw natural gas stream feed to the demethanizer column.

Element 9: Methods and systems wherein one or more components comprises one or more redundant components in the pressure management sub-system.

Element 10: Methods and systems configured to operate in modes selected from the group consisting of automatic continuous mode, automatic periodic mode, and manual mode.

Element 11: Methods and systems wherein one or more operational equipment are selected from the group consisting of pneumatic, electric, fuel, hydraulic, geothermal, and combinations thereof.

Element 12: Methods and systems comprising an HMI including a display with an interactive graphical user interface.

Element 13: Methods where the method described in Embodiment A (a first separator and pump downstream of demethanizer) is combined with the method described in Embodiment B (a second separator upstream of the demethanizer).

Element 14: Systems where the system described in Embodiment D (a first separator and pump downstream of demethanizer) is combined with the system described in Embodiment E (a second separator upstream of the demethanizer).

Element 15: Methods where the method described in Embodiment A (a first separator and pump downstream of demethanizer) is combined with the method described in Embodiment C (expander outlet bypasses the demethanizer).

Element 16: Systems where the system described in Embodiment D (a first separator and pump downstream of demethanizer) is combined with the system described in Embodiment F (expander outlet bypasses the demethanizer).

Element 17: Systems and methods wherein the demethanizer column is selected from a column configured to operate only as a demethanizer column, a column configured to operate alternatively as a demethanizer or a deethanizer, and a column configured to operate only as a deethanizer (which also removes methane).

In sum, at least three systems and methods are presented, including a first system comprising:

(a) a natural gas liquids (NGL) recovery unit;

(b) a nitrogen rejection unit (NRU); and

(c) a pressure management sub-system (PMSS) operatively and fluidly connecting the NGL recovery unit and the NRU, the PMSS comprising a set of conduits, individual members of the set of conduits fluidly connecting:
(i) a demethanizer column overhead to one or more heat exchangers and then to a separator,
(ii) the separator to a pump, the pump having a pump outlet;
(iii) the pump outlet with a lower section of an NRU distillation column;
(iv) the NRU distillation column bottoms to an expansion valve;
(v) the expansion valve with the one or more heat exchangers;
(vi) the separator overhead to a point of the fifth conduit downstream of the expansion valve and upstream of the one or more heat exchangers; and
(vii) the point of the fifth conduit to the one or more heat exchangers and then to an NGL recovery unit heat exchanger network.

In certain first systems the set of conduits may comprise:
(i) a first conduit fluidly connecting the demethanizer column overhead to the one or more heat exchangers and then to the separator;
(ii) a second conduit fluidly connecting the separator with the pump;
(iii) a third conduit fluidly connecting the pump outlet with the lower section of the NRU distillation column;
(iv) a fourth conduit fluidly connecting the NRU distillation column bottoms to the expansion valve;
(v) a fifth conduit fluidly connecting the expansion valve to the one or more heat exchangers;
(vi) a sixth conduit fluidly connecting the separator overhead with the NRU distillation column bottoms downstream of the expansion valve and upstream of the one or more heat exchangers; and
(vii) a seventh conduit configured to route the natural gas product to a NGL recovery unit heat exchanger network.

A method of producing a natural gas product using the first system may comprise:
feeding raw natural gas at ambient temperature into the NGL recovery unit, cooling and expanding the raw natural gas prior to feeding it, either as one stream or split into two or more streams, to the demethanizer column, while managing the pressure relationship between the demethanizer column and the NRU distillation column, routing the demethanizer column overhead to one or more heat exchangers and then to a separator, pumping a separator bottoms stream into the lower section of the NRU distillation column; forming an expanded NRU distillation column bottoms stream using the expansion valve, and combining the expanded stream with any separator vapor stream to form the natural gas product and routing the natural gas product through the one or more heat exchangers in the NRU and then to the NGL recovery unit heat exchanger network to warm the natural gas product to ambient temperature.

A second system comprises:

(a) a natural gas liquids (NGL) recovery unit;

(b) a nitrogen recovery unit (NRU); and

(c) a pressure management sub-system (PMSS) operatively and fluidly connecting the NGL recovery unit and the NRU, the PMSS comprising a set of conduits, individual members of the set of conduits fluidly connecting:
(i) an expander outlet to a separator, the separator having first and second outlets;
(ii) the first outlet of the separator with a first inlet of one or more heat exchangers, and a first outlet of the one or more heat exchangers with the lower section of an NRU distillation column;
(iii) a bottoms outlet of the NRU distillation column with an expansion valve;
(iv) the expansion valve with a second inlet of the one or more heat exchangers, and a second outlet of the one or more heat exchangers with the second outlet of the separator;
(v) the second outlet of the one or more heat exchangers with the separator second outlet to then feed a middle feed section of an NGL recovery unit demethanizer column; and
(vi) a demethanizer column overhead with a first receiver;
(vii) demethanizer column bottoms with a second receiver; wherein the (vi) and (vii) fluid connections avoid the NRU distillation column.

Certain second systems may comprise:
(i) a first conduit configured to route an expander outlet stream to a separator, forming a separator substantially vapor stream and a separator substantially liquid stream;
(ii) a second conduit configured to route the separator substantially vapor stream from the separator through one or more heat exchangers and then to
(iii) a third conduit to route the stream into a lower section of the NRU distillation column;
(iv) a fourth conduit configured to route NRU distillation column bottoms to an expansion valve, forming the expanded distillation column bottoms;
(v) a fifth conduit configured to route the expanded NRU distillation column bottoms through the one or more heat exchangers prior to combining with the separator substantially liquid stream;
(vi) a sixth conduit configured to combine the separator substantially liquid stream with the expanded NRU distillation column bottoms leaving the one or more heat exchangers to form a demethanizer feed stream;
(vii) a seventh conduit configured to route the demethanizer feed stream to a middle feed location of the demethanizer column,
(viii) an eighth conduit fluidly connecting demethanizer column overhead with a first receiver; and
(ix) a ninth conduit fluidly connecting demethanizer column bottoms with a second receiver, where the eighth and ninth conduits fluidly connect their respective receivers without going through the NRU distillation column.

A method of producing a natural gas product using the second system may comprise:
feeding raw natural gas at ambient temperature into the NGL recovery unit, cooling and expanding the raw natural gas prior to feeding it to the separator, while managing the pressure relationship between the demethanizer column and the NRU distillation column, feeding a separator vapor stream to the one or more heat exchangers in the NRU and then to the NRU distillation column; forming an expanded NRU distillation column bottoms stream using the expansion valve, and then warming the expanded NRU distillation column bottoms in the one or more heat exchangers in the NRU, and then combining the expanded stream with the separator liquid stream to form a combined feed steam to the demethanizer column; and warming the demethanizer overhead stream to ambient temperature to form the natural gas product.

A third system comprises:

(a) a natural gas liquids (NGL) recovery unit;

(b) a nitrogen rejection unit (NRU); and

(c) a pressure management sub-system (PMSS) operatively and fluidly connected to the NGL recovery unit and the NRU, the PMSS comprising a set of conduits, individual members of the set of conduits fluidly connecting:
(i) an expander outlet with a first inlet to one or more heat exchangers and then to a lower section of an NRU distillation column;
(ii) NRU distillation column bottoms with an expansion valve;
(iii) the expansion valve with a second inlet to the one or more heat exchangers;
(iv) the second outlet of the one or more heat exchangers with a middle location of an NGL recovery unit demethanizer column;
(v) a demethanizer column overhead with a first receiver;
(vi) demethanizer column bottoms with a second receiver; wherein the (v) and (vi) fluid connections avoid the NRU distillation column.

Certain third systems may comprise:
(i) a first conduit configured to route an expander outlet liquid and vapor through the one or more heat exchangers;
(ii) a second conduit fluidly connecting the one or more heat exchangers with a lower section of the NRU distillation column;
(iii) a third conduit configured to route NRU distillation column bottoms to the expansion valve, forming and expanded distillation column bottoms;
(iv) a fourth conduit configured to route the expanded distillation column bottoms through the one or more heat exchangers;
(v) a fifth conduit configured to route the expanded NRU distillation column bottoms leaving the one or more heat exchangers to feed a middle location of the demethanizer column;
(vi) a sixth conduit configured to route demethanizer column overhead to a first receiver; and
(vii) a seventh conduit configured to route demethanizer column bottoms to a second receiver, wherein the sixth and seventh conduits fluidly connect their respective receivers without going through the NRU distillation column.

A method of producing a natural gas product using the third system may comprise:
feeding raw natural gas at ambient temperature into the NGL recovery unit, cooling and expanding at least a portion of the raw natural gas, while managing the pressure relationship between the demethanizer column and the NRU distillation column, then feeding the expanded raw natural gas into the NRU distillation column; forming an expanded NRU distillation column bottoms stream using the expansion valve, and then warming the expanded NRU column bottoms in one or more heat exchangers in the NRU prior to feeding to the demethanizer column; and warming the demethanizer column overhead stream to ambient temperature to form the natural gas product.

From the foregoing detailed description of specific embodiments, it should be apparent that patentable systems, combinations, and methods have been described. Although specific embodiments of the disclosure have been described herein in some detail, this has been done solely for the purposes of describing various features and aspects of the methods and systems and is not intended to be limiting with respect to their scope. It is contemplated that various substitutions, alterations, and/or modifications, including but not limited to those implementation variations which may have been suggested herein, may be made to the described embodiments without departing from the scope of the appended claims. For example, one modification would be to take an existing NGL recovery unit/NRU combination and modify it to include a pressure management sub-system of this disclosure. Certain methods and systems of this disclosure may be devoid of certain steps, components and/or features: for example, systems devoid of NRU distillation unit feed pumps; systems devoid of demethanizer feed separators; systems devoid of low-strength steels; systems devoid of threaded fittings; systems devoid of welded fittings; methods devoid of a separation step upstream of the demethanizer methods devoid of a pump upstream of the NRU distillation column.

1. A system comprising:
(a) a natural gas liquids (NGL) recovery unit;
(b) a nitrogen rejection unit (NRU); and
(c) a pressure management sub-system (PMSS) operatively and fluidly connecting the NGL recovery unit and the NRU, the PMSS comprising a set of conduits, individual members of the set of conduits fluidly connecting:
(i) a demethanizer column overhead to one or more heat exchangers and then to a separator,
(ii) the separator to a pump, the pump having a pump outlet;
(iii) the pump outlet with a lower section of an NRU distillation column;
(iv) the NRU distillation column bottoms to an expansion valve;
(v) the expansion valve with the one or more heat exchangers;
(vi) the separator overhead to a point of the fifth conduit downstream of the expansion valve and upstream of the one or more heat exchangers; and
(vii) the point of the fifth conduit to the one or more heat exchangers and then to an NGL recovery unit heat exchanger network.

2. The system of claim 1 wherein the separator and the pump are configured so that the demethanizer column may operate at a pressure selected from the group consisting of lower than, equal to, or just above a pressure of the NRU distillation column.

3. The system of claim 1 wherein the NGL recovery unit heat exchanger network comprises at least one raw natural gas cooling heat exchanger and at least one separator configured to form at least one sub-cooled raw natural gas stream feed to the demethanizer column.

4. The system of claim 1 wherein the pressure management sub-system comprises one or more redundant components selected from the group consisting of two or more pumps connected in series and/or parallel configuration, two or more separators connected in series and/or parallel configuration, and two or more expansion valves connected in series and/or parallel configuration, or combinations thereof.

5. (canceled)

6. (canceled)

7. (canceled)

8. (canceled)

9. A system comprising:
(a) a natural gas liquids (NGL) recovery unit;
(b) a nitrogen recovery unit (NRU); and
(c) a pressure management sub-system (PMSS) operatively and fluidly connecting the NGL recovery unit and the NRU, the PMSS comprising a set of conduits, individual members of the set of conduits fluidly connecting:
(i) an expander outlet to a separator, the separator having first and second outlets;
(ii) the first outlet of the separator with a first inlet of one or more heat exchangers, and a first outlet of the one or more heat exchangers with an NRU distillation column;
(iii) a bottoms outlet of the NRU distillation column with an expansion valve;
(iv) the expansion valve with a second inlet of the one or more heat exchangers;
(v) the second outlet of the one or more heat exchangers with\hat{ } the separator second outlet to feed a middle feed section of an NGL recovery unit demethanizer column; and
(vi) a demethanizer column overhead with a first receiver;
(vii) demethanizer column bottoms with a second receiver; wherein the (vi) and
(vii) fluid connections avoid the NRU distillation column.

10. The system of claim 9 wherein the separator is configured so that the demethanizer column may operate at a pressure selected from the group consisting of lower than, equal to, or just above the NRU distillation column.

11. The system of claim 9 wherein the NGL recovery unit includes at least one raw natural gas cooling heat exchanger and at least one separator for forming at least one sub-cooled raw natural gas stream feed to the demethanizer column.

12. The system of claim 9 wherein the pressure management sub-system comprises one or more redundant components selected from the group consisting of two or more separators connected in series and/or parallel configuration, and two or more expansion valves connected in series and/or parallel configuration, and combinations thereof.

13. (canceled)

14. (canceled)

15. A system comprising:
(a) a natural gas liquids (NGL) recovery unit;
(b) a nitrogen rejection unit (NRU); and
(c) a pressure management sub-system (PMSS) operatively and fluidly connected to the NGL recovery unit and the NRU, the PMSS comprising a set of conduits, individual members of the set of conduits fluidly connecting:
(i) an expander outlet with a first inlet to one or more heat exchangers and then a lower section of an NRU distillation column;
(ii) NRU distillation column bottoms with an expansion valve;
(iii) the expansion valve with a second inlet to the one or more heat exchangers;
(iv) the second outlet of the one or more heat exchangers with a middle location of an NGL recovery unit demethanizer column;
(v) a demethanizer column overhead with a first receiver;
(vi) demethanizer column bottoms with a second receiver;
wherein the (v) and (vi) fluid connections avoid the NRU distillation column.

16. The system of claim 15 wherein the set of conduits and the expansion valve are configured so that the demethanizer column may operate at a pressure selected from the group consisting of lower than, equal to, or just above the NRU distillation column.

17. The system of claim 15 wherein the NGL recovery unit includes at least one raw natural gas cooling heat exchanger and at least one separator for forming at least one sub-cooled raw natural gas stream feed to the demethanizer column.

18. The system of claim 15 wherein the pressure management sub-system comprises one or more redundant components selected from the group consisting of two or more expansion valves connected in series and/or parallel configuration.

19. (canceled)

20. (canceled)

21. A method of producing a natural gas product using the system of claim 1, comprising: feeding raw natural gas at ambient temperature into the NGL recovery unit, cooling and expanding the raw natural gas prior to feeding it, either as one stream or split into the two or more streams, to the demethanizer column, while managing the pressure relationship between the demethanizer column and the NRU distillation column, routing the demethanizer column overhead to one or more heat exchangers and then to a separator; pumping a separator bottoms stream into the lower section of the NRU distillation column; forming an expanded NRU distillation column bottoms stream using the expansion valve, and combining the expanded stream with any separator vapor stream to form the natural gas product and routing the natural gas product through the one or more heat exchangers in the NRU and then to the NGL recovery unit heat exchanger network to warm the natural gas product to ambient temperature.

22. A method of producing a natural gas product using the system of claim 9, comprising: feeding raw natural gas at ambient temperature into the NGL recovery unit, cooling and expanding at least a portion of the raw natural gas prior to feeding it to the separator, while managing the pressure relationship between the demethanizer column and the NRU distillation column, feeding the separator vapor stream to the one or more heat exchangers in the NRU and then to the NRU distillation column; forming an expanded NRU distillation column bottoms stream using the expansion valve, and then warming the expanded NRU distillation column bottoms in the one or more heat exchangers in the NRU, and then combining the expanded stream with the separator liquid stream to form a combined feed stream to the demethanizer column; and warming the demethanizer column overhead stream to ambient temperature to form the natural gas product.

23. A method of producing a natural gas product using the system of claim 15, comprising: feeding raw natural gas at ambient temperature into the NGL recovery unit, cooling and expanding at least a portion of the raw natural gas, while managing the pressure relationship between the demethanizer column and the NRU distillation column, then feeding the expanded raw natural gas into the NRU distillation column; forming an expanded NRU distillation column bottoms stream using the expansion valve, and then warming the expanded NRU column bottoms in one or more heat exchangers in the NRU prior to feeding into the demethanizer column; and warming the demethanizer column overhead stream to ambient temperature to form the natural gas product.
